(19) United States
(12) Patent Application Publication
(10) Pub. No.: US 2009/0062338 A1
(43) Pub. Date: Mar. 5, 2009

(54) NITROXIDES FOR USE IN TREATING OR PREVENTING CARDIOVASCULAR DISEASE

(76) Inventors: Louis Habash, Newport Beach, CA (US); Clarence Jones, Huntington Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/815,446

(22) PCT Filed: Feb. 2, 2006
(86) PCT No.: PCT/US2006/003975

§ 371 Date: Sep. 3, 2008

Related U.S. Application Data
(60) Provisional application No. 60/649,254, filed on Feb. 2, 2005.

(51) Int. Cl. A61K 31/445; A61K 31/421; A61K 31/40

(52) U.S. Cl. 514/315; 514/374; 514/408

(57) ABSTRACT

Pharmaceutical compositions are provided that are useful in treating cardiovascular disease. The compositions comprise a pharmaceutically acceptable carrier, and an effective therapeutic or prophylactic amount of a nitroxide antioxidant that alters the expression of one or more genes related to the cardiovascular disease. Methods are also provided for the use of the pharmaceutical compositions in the treatment or prevention of cardiovascular disease. In a preferred embodiment, the nitroxide antioxidant is Tempol (4-hydroxy-2,2,6,6-tetramethylpiperidine-1-oxyl), and the cardiovascular disease is myocardial infarction.

### BACKGROUND OF THE INVENTION

1. Field of the Invention

The present invention relates to pharmaceutical compositions useful for treating or preventing cardiovascular disease, and to methods for using these compositions in treating or preventing cardiovascular disease.

2. Description of the Related Art

Cardiovascular disease is common in industrialized countries; for example, approximately 1.1 million acute myocardial infarctions (AMI) occur every year in the United States. A common cause of AMI is atherosclerosis of the coronary arteries. AMI leads to myocardial damage as a result of myocardial ischemia. This ischemia can lead to the triggering of apoptotic mechanisms that cause cell death.

AMI exhibits a mortality rate of approximately 30%; more than half of these deaths occur before the patient reaches the hospital. Of those patients who survive the initial hospitalization, approximately 1 in 25 will die in the first year after suffering an AMI, and elderly patients fare even worse, with 30% dying within one year of an AMI.

Part of the reason for this high rate of mortality in patients who initially survive AMI is the ventricular dysfunction that often follows the initial event. Termed “ventricular remodeling,” this involves the dilation of the left ventricle, initially from expansion of the infarct with resulting thinning and elongation of the infarct region, and subsequently from lengthening of noninfarcted segments as a result of an architectural rearrangement of the myocardium, including cardiomyocytes and interstitial cells. In cases of large AMI, this progressive dilation often results in increasing hemodynamic impairment, more frequent progression to heart failure, and a poor prognosis.

It would be desirable to develop methods to avoid the progression from atherosclerosis to AMI, and to avoid these severe consequences of AMI.

Gene therapy offers a potential alternative for the treatment of cardiovascular disease, especially the treatment of AMI. To this end, it would be desirable to identify genes related to cardiovascular disease and develop methods of altering the expression patterns of those genes so as to prevent the development of the disease or reduce its effects once it has occurred.

### SUMMARY OF THE INVENTION

Pharmaceutical compositions are provided that are useful in preventing and treating cardiovascular disease. The compositions comprise a pharmaceutically acceptable carrier, and an effective therapeutic or prophylactic amount of an agent that changes the expression pattern of a gene related to cardiovascular disease. Methods are also provided for the use of the pharmaceutical compositions in the alteration of intracellular levels of cardiovascular disease-related proteins. In a preferred embodiment, the agent is a nitroxide antioxidant, such as Tempol (4-hydroxy-2,2,6,6-tetramethylpiperidine-1-oxyl). Use of nitroxide antioxidants such as Tempol has been proposed as a treatment for ischemic cell damage in cases of acute resuscitation, such as myocardial infarction. However, the use of Tempol as a treatment after the primary ischemic situation has been resolved, in order to ameliorate the longer-term effects of cardiovascular disease by reducing ventricular remodeling, has not been proposed.

### DETAILED DESCRIPTION OF THE PREFERRED EMBODIMENT

As described above, a composition and method are disclosed which are useful in treating or preventing cardiovascular disease. In a preferred embodiment, the agent used to downregulate genes related to cardiovascular disease is a nitroxide antioxidant. Tempol is a stable nitroxide radical characterized by the chemical formula 4-hydroxy-2,2,6,6-tetramethylpiperidine-1-oxyl that has antioxidative properties. The present applicants have discovered that Tempol also possesses the novel property of altering the expression of genes encoding for proteins associated with the development or progression of cardiovascular disease (see Tables 1 and 2 below). Previous therapies have generally not focused on altering the expression patterns of such cardiovascular disease-related genes.

Tempol accordingly affects the upstream source of implicated proteins by altering the expression of cardiovascular disease-associated genes.

The use of other nitroxide compounds is also contemplated. According to certain embodiments the nitroxide compound can be selected from the following formulas:

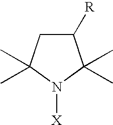

Wherein X is selected from O. and OH, and R is selected from COOH, CONH, CN, and CH₂NH₂.

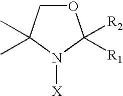

Wherein X is selected from O. and OH, and R₁ is selected from CH₃ and spirocyclohexyl, and R₂ is selected from C₂H₅ and spirocyclohexyl.

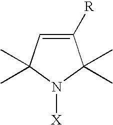

Wherein X is selected from O. and OH and R is selected from CONH.

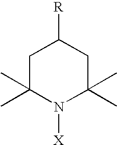

Wherein X is selected from O. and OH and R is selected from H, OH, and NH₂.

Suitable nitroxide compounds can also be found in Proctor, U.S. Pat. No. 5,352,442, and Mitchell et al., U.S. Pat. No. 5,462,946, both of which are hereby incorporated by reference in their entireties.

A non-limiting list of nitroxide compounds include: 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, and the like.

### Experimental Protocol

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that a gene related to cardiovascular disease, hepatocyte growth factor, exhibited a more than threefold increase in expression. This gene is shown in Table 1.

TABLE 1
CARDIOVASCULAR DISEASE-RELATED GENE EXHIBITING
INCREASED EXPRESSION IN CARDIAC TISSUE
AFTER TEMPOL ADMINISTRATION

| | De- | | | | TEMPOL- | | | |
|---|---|---|---|---|---|---|---|---|
| | scrip- | Control mice | | | treated mice | | | Fold |
| ORF | tion | tpc1 | tpc2 | tpc3 | tp51 | tp52 | tp53 | change |

UPREGULATED GENE

| | | | | | | | | |
|---|---|---|---|---|---|---|---|---|
| W12681 | Hepato- | 97 | 141 | 15 | 152 | 151 | 207 | 3.2 |
| | cyte | | | | | | | |
| | Growth | | | | | | | |
| | Factor | | | | | | | |

In a further gene expression study, Tempol was administered to experimental mice at a dose of 5 g/kg of diet from 12 months through 15 months. Mice receiving the same diet without the addition of Tempol were used as a negative control. At the age of 15 months, the adipose tissue of the experimental animals was obtained. The expression of a broad spectrum of genes in the adipose tissue was assessed using chip-based microarray technology. Specifically, in this case an Affymetrix MOE430A 2.0 array, containing 12,960 genes, was employed. Such chips are well known in the art and are widely used to assess gene expression. The experimental results on the adipose tissue show that genes related to cardiovascular disease, caspase 3 and adiponectin, exhibited altered expression. These genes are shown in Table 2.

TABLE 2
CARDIOVASCULAR DISEASE-RELATED GENES EXHIBITING
ALTERED EXPRESSION IN ADIPOSE TISSUE AFTER
TEMPOL ADMINISTRATION

| | Mean | Mean (Tempol- | | |
|---|---|---|---|---|
| Description | (Control mice) | treated mice) | P Value | Fold change |
| Adiponectin, | 27698 | 33876 | 0.003 | 1.22 |
| C1Q and | | | | |
| collagen | | | | |
| domain | | | | |
| containing | | | | |
| Caspase 3 | 604 | 414 | 0 | −1.47 |

A short summary of the gene described in Tables 1 and 2 is provided below.

### Hepatocyte Growth Factor (HGF)

HGF has been implicated in tissue regeneration, angiogenesis and apoptosis. In a recent study, mice were given adenovirus encoding human HGF intramuscularly three days following the induction of myocardial infarction, while in a control group, the LacA gene was used. (Li et al., Circulation 107:2499-506 (2003).) The mice receiving the human HGF gene showed persistently increased plasma HGF, and improved left ventricular remodeling and less cardiac dysfunction was also observed at four weeks post-treatment, as indicated by a smaller left ventricular cavity and heart/body weight ratio, greater percent fractional shortening and left ventricular ±dP/dt, and lower left ventricular end-diastolic pressure. The cardiomyocytes proximate to the myocardial infarct in the treated mice were also greatly hypertrophied, and the infarct wall was thicker due to an increased density of both cardiomyocytes and blood vessels.

As shown in Table 1, the expression of HGF in the cardiac tissue of the experimental mice was increased 3.2-fold in the animals treated with Tempol.

### Adiponecton (ADIPOQ)

ADIPOQ is an adipocyte-derived peptide that regulates energy metabolism and endothelial activation. ADIPOQ levels have been shown to be decreased in patients with cardiovascular disease. For example, a recent study showed that a population of patients with arteriosclerosis obliterans, a typical disorder of arteriosclerosis in which arterial obstruction occurs in arteries supplying blood in lower extremities, had a significantly lower level of serum adiponectin than that found in control subjects (Iawano et al., Metabolism Clinical and Experimental 54 (2005) 653-656). A further study of patients with coronary artery disease showed that patients with the disease had a significantly lower level of adiponectin as compared with the control subjects; the study found a 63% reduction in risk of coronary artery disease per 6.3 μg/ml increase in serum adiponectin levels (Costacou et al., Diabetologia 48 (2005) 41-48). Another recent study found that angiotensin II infused into experimental rats produced vascular endothelial dysfunction and a decrease in circulating adiponectin. The deleterious effects of angiotensin II on the endothelium were found to be ameliorated by the introduction of Tempol, which effect was ascribed to a restoration of nitric oxide bioactivity (Hattori et al., Diabetalogia 48 (2005) 1066-1074.) However, it is possible that part of the effect of the introduction of Tempol was due to an upregulation of the adiponectin gene.

As shown in Table 2, the expression of adiponectin in the adipose tissue of the experimental mice was increased 1.22-fold in the animals treated with Tempol.

### Caspase 3

Caspase 3 is a member of the cystene-aspartic acid (caspase) family of proteases. Caspases exist as inactive proenzymes which undergo proteolytic processing and dimerization to form the active enzymes, and are activated in a sequential manner and play a central role in the execution phase of apoptotic death by cleaving many structural and regulatory proteins. The proapoptotic proteases are organized in a hierarchical cascade: the apical or initiator caspases 8 and 9 cleave and activate the effector caspases 3, 6, and 7. Caspase 3 has been shown to be the primary executioner caspase. Mice engineered to overexpress caspase 3 in the heart have been shown to have an increased susceptibility to death when subjected to cardiac ischemia-reperfusion injury: only 45% and 30% of caspase 3-overexpressing mice were alive after 2 and 24 hours of reperfusion, respectively, versus 90% and 70% of control mice (Condorelli et al., PNAS 98:17 (2001) 9977-9982). The infarct size of the caspase 3 overexpressing mice was also increased.

As shown in Table 3, the expression of caspase 3 in the adipose tissue of the experimental mice was decreased 1.47-fold in the animals treated with Tempol.

### Preferred Embodiment

### Cardiovascular Disease Prophylaxis and Treatment Protocol

As described above, Tempol has the effect of altering the expression of genes related to cardiovascular disease. Since the expression of these genes is altered, administration of Tempol will have a beneficial effect by altering concentrations of gene products that are linked to the amelioration of cardiovascular disease. Specifically, Tempol will have at least the beneficial effects of increasing HGF and ADIPOQ concentrations and thereby reducing the extent of ventricular remodeling and the risk of developing cardiovascular disease, and of reducing the concentration of caspase 3 and thereby reducing the extent of apoptosis subsequent to ischemia-reperfusion injury. In a preferred embodiment of the present invention, therefore, Tempol is administered to a mammalian host, such as a human, exhibiting no symptoms of cardiovascular disease in order to prevent the development of cardiovascular disease. Particularly preferred patients are those who are predisposed or otherwise at risk for cardiovascular disease, such as those with a family history of cardiovascular disease, those with genetic or serum markers associated with cardiovascular disease, or those scheduled to undergo medical procedures in which cardiovascular disease is a possible side effect. Alternatively, Tempol may be administered to a human exhibiting cardiovascular disease, such as post-AMI, after primary treatment of ischemia has been conducted, in order to improve the recovery of the patient from the cardiovascular disease. For this purpose, Tempol, non-toxic salts thereof, acid addition salts thereof or hydrates thereof may be administered systemically or locally, usually by oral or parenteral administration.

The doses to be administered are determined depending upon, for example, age, body weight, symptom, the desired therapeutic effect, the route of administration, and the duration of the treatment. In the human adult, the dose per person at a time is generally from about 0.01 to about 1000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time is generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration is also contemplated for from 1 to 24 hours per day to achieve a target concentration from about 0.01 mg/L to about 100 mg/L. Specific examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does, however, depend upon various conditions, and there may be cases wherein doses lower than or greater than the ranges specified above are used.

Tempol may be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, Tempol is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. In injections, Tempol may be dissolved, suspended and emulsified in a solvent. The solvents are, for example, distilled water for injection, physiological salt solution, vegetable oil, propylene glycol, polyethylene glycol, alcohol such as ethanol, or a mixture thereof. Moreover the injections may also include stabilizing agents, agents to assist dissolution (e.g. glutamic acid, aspartic acid or POLYSORBATE80 (registered trade mark)), suspending agents, emulsifying agents, soothing agents, buffer agents, preserving agents, etc. They are sterilized in the final process or manufactured and prepared by sterile procedure. They may also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise Tempol and are administered by methods known in the art.

Spray compositions may comprise additional substances other than diluents: e.g. stabilizing agents (e.g. sodium sulfite hydride), isotonic buffers (e.g. sodium chloride, sodium citrate or citric acid). For preparation of such spray compositions, for example, the method described in U.S. Pat. No. 2,868,691 or No. 3,095,355 may be used. Briefly, a small aerosol particle size useful for effective distribution of the medicament may be obtained by employing self-propelling compositions containing the drugs in micronized form dispersed in a propellant composition. Effective dispersion of the finely divided drug particles may be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size is less than about 5 microns. The propellant composition may employ, as the suspending agent, a fatty alcohol such as oleyl alcohol. The minimum quantity of suspending agent is approximately 0.1 to 0.2 percent by weight of the total composition. The amount of suspending agent is preferably less than about 4 percent by weight of the total composition to maintain an upper particle size limit of less than 10 microns, and preferably 5 microns. Propellants that may be employed include hydrofluoroalkane propellants and chlorofluorocarbon propellants. Dry powder inhalation may also be employed.

### Example 1

A 70-kilogram patient three days post myocardial infarction is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein level of hepatocyte growth factor and adiponectin in the plasma is increased, and the protein level of caspase 3 is decreased.

### Example 2

A 70-kilogram patient at risk for myocardial infarction is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein level of hepatocyte growth factor and adiponectin in the plasma is increased, and the protein level of caspase 3 is decreased.

1-3. (canceled)

4. A method, for altering intracellular levels of one or more proteins associated with cardiovascular disease resulting from myocardial infarction, comprising:
identifying an individual in need of altering levels of the cardiovascular disease-associated proteins; and
administering to that individual an effective amount of a nitroxide antioxidant selected from the group consisting of compounds having the following formulas: 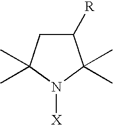
wherein X is selected from O. and OH, and R is selected from COOH, CONH, CN, and CH₂NH₂; 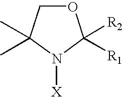
wherein X is selected from O. and OH, and R₁ is selected from CH₃ and spirocyclohexyl, and R₂ is selected from C₂H₅ and spirocyclohexyl; 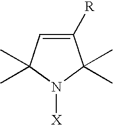
wherein X is selected from O. and OH and R is selected from CONH; 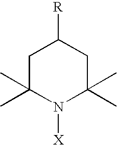
wherein X is selected from O. and OH and R is selected from H, OH, and NH₂, the level of the cardiovascular disease-associated protein is increased, and the cardiovascular disease-associated protein is hepatocyte growth factor or adiponectin.

5. A method for altering intracellular levels of one or more proteins associated with cardiovascular disease resulting from myocardial infarction, comprising:
identifying an individual in need of altering levels of the cardiovascular disease-associated proteins; and
administering to that individual an effective amount of a nitroxide antioxidant selected from the group consisting of compounds having the following formulas: 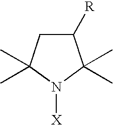
wherein X is selected from O. and OH, and R is selected from COOH, CONH, CN, and CH₂NH₂; 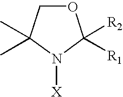
wherein X is selected from O. and OH, and R₁ is selected from CH₃ and spirocyclohexyl, and R₂ is selected from C₂H₅ and spirocyclohexyl; 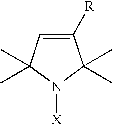
wherein X is selected from O. and OH and R is selected from CONH; 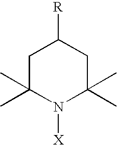
wherein X is selected from O. and OH and R is selected from H, OH, and NH₂, and wherein the level of the cardiovascular disease associated protein is decreased.

6. The method of claim 5, wherein the cardiovascular disease associated protein is caspase 3.

7. A method for altering intracellular levels of one or more proteins associated with cardiovascular disease resulting from myocardial infarction, comprising:
identifying an individual in need of altering levels of the cardiovascular disease-associated proteins; and
administering to that individual an effective amount of a nitroxide antioxidant selected from the group consisting of compounds having the following formulas: 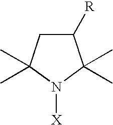
wherein X is selected from O. and OH, and R is selected from COOH, CONH, CN, and CH₂NH₂; 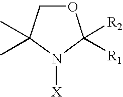
wherein X is selected from O. and OH, and R₁ is selected from CH₃ and spirocyclohexyl, and R₂ is selected from C₂H₅ and spirocyclohexyl; 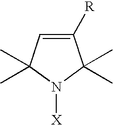
wherein X is selected from O. and OH and R is selected from CONH; 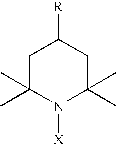
wherein X is selected from O. and OH and R is selected from H, OH, and NH₂, and wherein the cardiovascular disease is selected from the group consisting of ventricular remodeling and apoptosis of cardiac tissue.

8-16. (canceled)

17. A method for inhibiting the progresssion of cardiovascular disease that results from myocardial infarction and is associated with a protein, comprising:
identifying an individual affected by or at risk for the protein-associated cardiovascular disease; and
administering to that individual an amount of a nitroxide antioxidant effective to alter expression of a gene associated with the protein-associated cardiovascular disease, wherein the nitroxide antioxidant is selected from the group consisting of compounds having the following formulas: 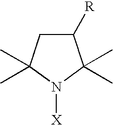
wherein X is selected from O. and OH, and R is selected from COOH, CONH, CN, and CH₂NH₂; 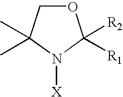
wherein X is selected from O. and OH, and R₁ is selected from CH₃ and spirocyclohexyl, and R₂ is selected from C₂H₅ and spirocyclohexyl; 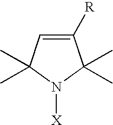
wherein X is selected from O. and OH and R is selected from CONH; 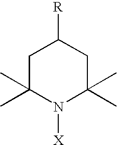
wherein X is selected from O. and OH and R is selected from H, OH, and NH₂, and
where the expression of the gene is increased.

18. The method of claim 17, wherein the gene is hepatocyte growth factor or adiponectin.

19. A method for inhibiting the progression of cardiovascular disease that results from myocardial infarction and is associated with a protein, comprising:
identifying an individual affected by or at risk for the protein-associated cardiovascular disease; and
administering to that individual an amount of a nitroxide antioxidant effective to alter expression of a gene associated with the protein-associated cardiovascular disease, wherein the nitroxide antioxidant is selected from the group consisting of compounds having the following formulas: 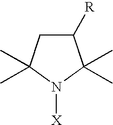
wherein X is select O. and OH, and R is selected from COOH, CONH, CN, and CH₂NH₂; 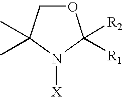
wherein X is selected from O. and OH, and R₁ is selected from CH₃ and spirocyclohexyl, and R₂ is selected from C₂H₅ and spirocyclohexyl; 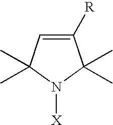
wherein X is selected from O. and OH and R is selected from CONH; 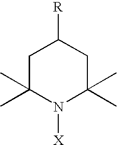
wherein X is selected from O. and OH and R is selected from H, OH, and NH₂, and
where the expression of the gene is decreased.

20. The method of claim 19, wherein the gene is caspase 3.

21. A method for inhibiting the progression of cardiovascular disease that results from myocardial infarction and is associated with a protein, comprising:
identifying an individual affected by or at risk for the protein-associated cardiovascular disease; and
administering to that individual an amount of a nitroxide antioxidant effective to alter expression of a gene associated with the protein-associated cardiovascular disease, wherein the nitroxide antioxidant is selected from the group consisting of compounds having the following formulas: 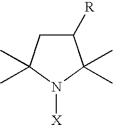
wherein X is selected from O. and OH, and R is selected from COOH, CONH, CN, and CH₂NH₂; 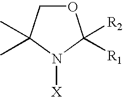
wherein X is selected from O. and OH, and R₁ is selected from CH₃ and spirocyclohexyl, and R₂ is selected from C₂H₅ and spirocyclohexyl; 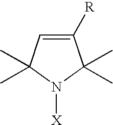
wherein X is selected from O. and OH and R is selected from CONH; 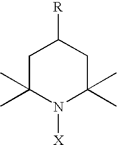
wherein X is selected from O. and OH and R is selected from H, OH, and NH₂, and
wherein the protein-associated cardiovascular disease is ventricular remodeling.

22. A method for inhibiting the progression of cardiovascular disease that results from myocardial infarction and is associated with a protein, comprising:
identifying an individual affected by or at risk for the protein-associated cardiovascular disease; and
administering to that individual an amount of a nitroxide antioxidant effective to alter expression of a gene associated with the protein-associated cardiovascular disease, wherein the nitroxide antioxidant is selected from the group consisting of compounds having the following formulas: 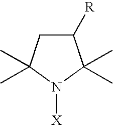
wherein X is selected from O. and OH, and R is selected from COOH, CONH, CN, and CH₂NH₂; 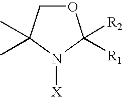
wherein X is selected from O. and OH, and R₁ is selected from CH₃ and spirocyclohexyl, and R₂ is selected from C₂H₅ and spirocyclohexyl; 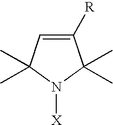
wherein X is selected from O. and OH and R is selected from CONH; 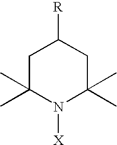
wherein X is selected from O. and OH and R is selected from H, OH, and NH₂, and
wherein the protein-associated cardiovascular disease is apoptosis of cardiac tissue.
